(19) United States
(12) Patent Application Publication
(10) Pub. No.: US 2016/0089033 A1
(43) Pub. Date: Mar. 31, 2016

(54) DETERMINING TIMING AND CONTEXT FOR CARDIOVASCULAR MEASUREMENTS

(71) Applicant: Microsoft Corporation, Redmond, WA (US)

(72) Inventors: T. Scott Saponas, Woodinville, WA (US); Dan Morris, Bellevue, WA (US); Nicolas Villar, Cambridge (GB); Shwetak Patel, Seattle, WA (US); Greg R. Smith, Bellevue, WA (US); Desney Tan, Kirkland, WA (US)

(21) Appl. No.: 14/500,615

(22) Filed: Sep. 29, 2014

(51) Int. Cl. A61B 5/0205; A61B 5/00; A61B 5/22; A61B 5/11

(52) U.S. Cl. CPC A61B 5/0205; A61B 5/1118; A61B 5/721; A61B 5/7285; A61B 5/7278; A61B 5/486; A61B 5/1112; A61B 5/0022; A61B 5/222; A61B 5/4806; A61B 5/4866; A61B 5/1123; A61B 5/7246; A61B 5/7282; A61B 5/7221; A61B 5/7275; A61B 5/681; A61B 5/742; A61B 5/02405

(57) ABSTRACT

The cardiovascular vital signs of a user are measured. One or more user activity metrics is received from one or more user activity sensors. A type of activity the user is currently engaged in is inferred from the received user activity metrics. Additional context that is associated with the inferred type of activity may also be identified. A determination is made as to if it is time to measure the cardiovascular vital signs of the user, where this determination is based on the inferred type of activity and may also be based on the identified additional context. Whenever it is determined to be time to measure the cardiovascular vital signs of the user, this measurement is made.

### BACKGROUND

As is appreciated in the arts of medicine and health, vital signs are indicators of the human body's most basic functions, and are useful in both detecting and monitoring medical conditions. The four main vital signs that are routinely measured by medical professionals and health care providers are body temperature, blood pressure, heart rate (also known as pulse rate), and respiration (i.e., breathing) rate. Blood pressure and heart rate are two of the various indicators of a person's cardiovascular health. When the human heart expels blood a pressure wave is generated. This pressure wave travels through one or more arteries in the human body, and is affected by the lining and the stiffness/compliance of the arteries. This pressure wave is also known as a pressure pulse.

Heart disease is the leading cause of death in the United States. For example, in 2011 heart disease accounted for approximately 600,000 deaths in the United States, which is nearly 31 percent of all the reported deaths in the United States in 2011. According to the Centers for Disease Control and Prevention, approximately one in three American adults suffers from high blood pressure, which is also known as and herein also referred to as hypertension. Hypertension is a well-understood risk factor for stroke, heart attack, heart failure, and arterial aneurysm. Hypertension is also the leading cause of renal failure. In the United States alone, it is estimated that hypertension directly results in billions of dollars in health care costs annually, and nearly 1,000 deaths daily. Unfortunately, hypertension has no visible warning signs or symptoms, and many people do not even realize they have it. This is particularly unfortunate because hypertension is treatable. For example, lifestyle changes such as diet and exercise are known to be effective in preventing the progression of hypertension. Additionally, numerous medications are available for treating hypertension.

### SUMMARY

Cardiovascular measurements technique implementations described herein are generally applicable to measuring the cardiovascular vital signs of a user. In one exemplary implementation one or more user activity metrics is received from one or more user activity sensors. A type of activity the user is currently engaged in is then inferred from the received user activity metrics. A determination is then made as to if it is time to measure the cardiovascular vital signs of the user, where this determination is based on the inferred type of activity. Then, whenever it is determined to be time to measure the cardiovascular vital signs of the user, this measurement is made.

It should be noted that the foregoing Summary is provided to introduce a selection of concepts, in a simplified form, that are further described below in the Detailed Description. This Summary is not intended to identify key features or essential features of the claimed subject matter, nor is it intended to be used as an aid in determining the scope of the claimed subject matter. Its sole purpose is to present some concepts of the claimed subject matter in a simplified form as a prelude to the more detailed description that is presented below.

### DESCRIPTION OF THE DRAWINGS

The specific features, aspects, and advantages of the cardiovascular measurements technique implementations described herein will become better understood with regard to the following description, appended claims, and accompanying drawings where:

FIG. 1 is a diagram illustrating one implementation, in simplified form, of an architectural framework for realizing the cardiovascular measurements technique implementations described herein.

FIG. 2 is a diagram illustrating another implementation, in simplified form, of an architectural framework for realizing the cardiovascular measurements technique implementations described herein.

FIG. 3 is a flow diagram illustrating one implementation, in simplified form, of a process for measuring the cardiovascular vital signs of a user.

FIGS. 4-8 are diagrams illustrating different scenarios, in simplified form, of exemplary types of activity a user is currently engaged in being inferred from received user activity metrics.

FIG. 9 is a flow diagram illustrating another implementation, in simplified form, of a process for measuring the cardiovascular vital signs of a user.

FIGS. 10 and 11 are a flow diagram illustrating yet another implementation, in simplified form, of a process for measuring the cardiovascular vital signs of a user.

FIG. 12 is a diagram illustrating an exemplary implementation, in simplified form, of a system framework for realizing the cardiovascular measurements technique implementations described herein.

FIG. 13 is a diagram illustrating a simplified example of a general-purpose computer system on which various implementations and elements of the cardiovascular measurements technique, as described herein, may be realized.

### DETAILED DESCRIPTION

In the following description of cardiovascular measurements technique implementations reference is made to the accompanying drawings which form a part hereof, and in which are shown, by way of illustration, specific implementations in which the cardiovascular measurements technique can be practiced. It is understood that other implementations can be utilized and structural changes can be made without departing from the scope of the cardiovascular measurements technique implementations.

It is also noted that for the sake of clarity specific terminology will be resorted to in describing the cardiovascular measurements technique implementations described herein and it is not intended for these implementations to be limited to the specific terms so chosen. Furthermore, it is to be understood that each specific term includes all its technical equivalents that operate in a broadly similar manner to achieve a similar purpose. Reference herein to “one implementation”, or “another implementation”, or an “exemplary implementation”, or an “alternate implementation”, or “one version”, or “another version”, or an “exemplary version”, or an “alternate version” means that a particular feature, a particular structure, or particular characteristics described in connection with the implementation or version can be included in at least one implementation of the cardiovascular measurements technique. The appearances of the phrases “in one implementation”, “in another implementation”, “in an exemplary implementation”, “in an alternate implementation”, “in one version”, “in another version”, “in an exemplary version”, and “in an alternate version” in various places in the specification are not necessarily all referring to the same implementation or version, nor are separate or alternative implementations/versions mutually exclusive of other implementations/versions. Yet furthermore, the order of process flow representing one or more implementations or versions of the cardiovascular measurements technique does not inherently indicate any particular order not imply any limitations of the cardiovascular measurements technique.

As utilized herein, the terms “component,” “system,” “client” and the like are intended to refer to a computer-related entity, either hardware, software (e.g., in execution), firmware, or a combination thereof. For example, a component can be a process running on a processor, an object, an executable, a program, a function, a library, a subroutine, a computer, or a combination of software and hardware. By way of illustration, both an application running on a server and the server can be a component. One or more components can reside within a process and a component can be localized on one computer and/or distributed between two or more computers. The term “processor” is generally understood to refer to a hardware component, such as a processing unit of a computer system.

Furthermore, to the extent that the terms “includes,” “including,” “has,” “contains,” variants thereof, and other similar words are used in either this detailed description or the claims, these terms are intended to be inclusive, in a manner similar to the term “comprising”, as an open transition word without precluding any additional or other elements.

### 1.0 Determining Timing and Context for Cardiovascular Measurements

The cardiovascular measurements technique implementations described herein are generally applicable to determining the timing and context for measuring the cardiovascular vital signs of a user. As described heretofore, vital signs are indicators of the human body's most basic functions, and are useful in both detecting and monitoring medical conditions. As will be described in more detail hereafter, the cardiovascular measurements technique implementations can measure various cardiovascular vital signs including, but not limited to, a pulse transit time, a pulse wave velocity, a heart rate (also known as a pulse rate), heart rate variability, a pulse waveform whose morphology can be analyzed to derive various other cardiovascular metrics, blood pressure, blood pressure variability, and any combination thereof. The measured cardiovascular vital signs can then be used in various ways, examples of which are provided hereafter.

The term “arterial site” is used herein to refer to any location on a user's body where movement in an underlying artery can be sensed. The term “portable computing device” is used herein to refer to a mobile computing device that can be attached to an arterial site on a user's body. Exemplary arterial sites to which the portable computing device can be attached are described in more detail hereafter. The portable computing device can be realized in various forms. In an exemplary implementation of the cardiovascular measurements technique described herein the portable computing device is realized in the form of a wearable computing device that is worn by the user on an arterial site.

The term “pulse transit time” is used herein to refer to the amount of time it takes for a pressure wave that is generated by blood being expelled from a user's heart each time the heart beats (which is hereafter sometimes simply referred to as a pulse pressure wave, and is also known as a pressure pulse) to travel through an artery (or series of arteries) from one arterial site on the user's body to another arterial site on the user's body. More particularly and as will be appreciated from the more detailed description that follows, in one version of the cardiovascular measurements technique implementations described herein where the cardiovascular vital signs of a user are measured using a portable computing device that is attached to (e.g., worn on) one of the user's wrists (which is an exemplary viable arterial site), pulse transit time refers to the amount of time it takes for the pulse pressure wave to travel from the user's heart, through an artery (or series of arteries), to the wrist upon which the portable computing device is attached. As is appreciated in the arts of medicine and cardiovascular health, there is a known correlation between pulse transit time and other cardiovascular metrics such as blood pressure, arterial compliance, and the hardening of artery walls. Although other body metrics (such as the user's height, their weight, their age, their gender, their race, and the actual arterial distance between the just-described two arterial sites on the user's body, among other types of body metrics) influence the user's blood pressure, the pulse transit time measurement that is made by the cardiovascular measurements technique implementations can be used to determine the user's blood pressure based on the just-described known correlation between pulse transit time and blood pressure.

The term “pulse wave velocity” is used herein to refer to the average speed at which a pulse pressure wave travels through an artery (or series of arteries) from one arterial site on the user's body to another arterial site on the user's body. More particularly and as will be appreciated from the more detailed description that follows, in the just-described version of the cardiovascular measurements technique implementations described herein where the cardiovascular vital signs of a user are measured using a portable computing device that is attached to one of the user's wrists, pulse wave velocity refers to the average speed at which the pulse pressure wave travels from the user's heart, through an artery (or series of arteries), to the wrist upon which the portable computing device is attached. As is appreciated in the arts of medicine and cardiovascular health, there is a known correlation between pulse wave velocity and cardiovascular diseases such as hypertension (also known as high blood pressure). More particularly, as a person ages their arteries generally get stiffer. This increasing arterial stiffness makes the person's heart work harder and also makes the pulse pressure wave travel faster through their arteries, thus increasing their risk of cardiovascular diseases such as hypertension.

As will be appreciated from the more detailed description that follows, the cardiovascular measurements technique implementations described herein are advantageous for various reasons such as the following. The cardiovascular measurements technique implementations provide users with a cost effective and easy to use assessment of their cardiovascular health. The cardiovascular measurements technique implementations can also prevent many heart-disease-related deaths by making users who have hypertension aware of it. Once a given user is made aware of their having hypertension, the cardiovascular measurements technique implementations can be used to routinely monitor the user's cardiovascular vital signs, and encourage the user to treat their hypertension by consulting with a doctor and making appropriate lifestyle changes. The cardiovascular measurements technique implementations also measure users' cardiovascular vital signs in a non-invasive and non-intrusive (e.g., a passive) manner. The cardiovascular measurements technique implementations thus allow users to routinely measure/monitor their cardiovascular vital signs without pain and discomfort.

As will also be appreciated from the more detailed description that follows, the cardiovascular measurements technique implementations described herein can be employed in various applications. The cardiovascular measurements technique implementations can also be realized in various types of computing devices and various computing architectures. The cardiovascular measurements technique implementations are also easy to operate, and are not restricted to being used by a trained medical technician or doctor in a controlled medical setting such as a laboratory or doctor's office. The cardiovascular measurements technique implementations thus allow users to conveniently and automatically measure their cardiovascular vital signs at one or more opportunistic times during the normal course of their day while they are either stationary or ambulatory, including while they are involved in or performing a wide variety of physical activities.

### 1.1 Architectural Framework

Various architectural frameworks can be used to realize the cardiovascular measurements technique implementations described herein. This section describes exemplary implementations of such architectural frameworks. It is noted that in addition to the architectural frameworks described in this section, many other types of architectural frameworks may also be used to realize the cardiovascular measurements technique implementations.

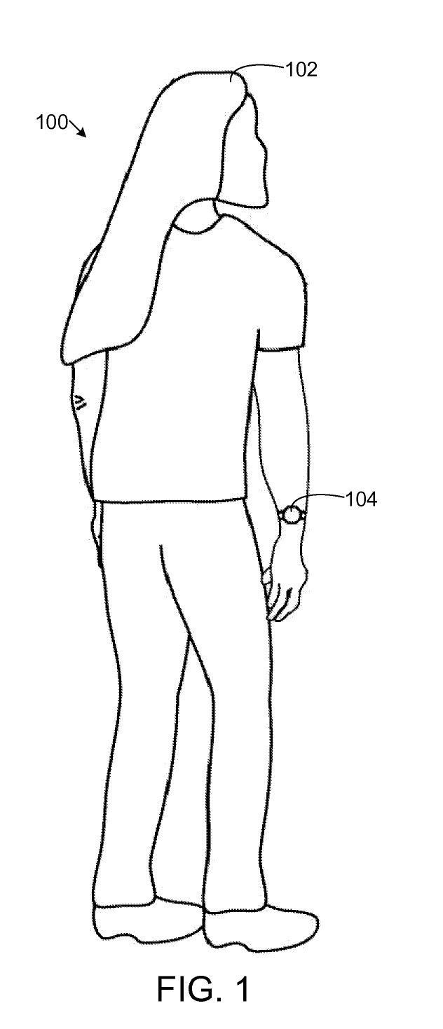

FIG. 1 illustrates one implementation, in simplified form, of an architectural framework for realizing the cardiovascular measurements technique implementations described herein. In the architectural framework 100 exemplified in FIG. 1, the cardiovascular measurements technique implementations are realized in the form of a portable computing device 104 that is attached to (e.g., worn on) one of the wrists of a user 102. The portable computing device 104 includes a set of user activity sensors (not shown), one or more primary electrocardiography (ECG) electrodes (not shown), and one or more secondary ECG electrodes (not shown). As will be described in more detail hereafter, the user activity sensors are used to detect various attributes of the user 102 and their current environment whenever the portable computing device 104 is attached to the user. The primary and secondary ECG electrodes are used to detect an ECG signal associated with the user 102 whenever the portable computing device 104 is attached to the user. In other words, the primary and secondary ECG electrodes are configured to measure an ECG signal associated with the user. The portable computing device 104 can optionally also include a touch-enabled display subsystem (not shown) via which information may be displayed to the user, and the user may enter information into the device. It is noted that various alternate implementations (not shown) of the architectural framework 100 are possible. For example, rather than the portable computing device 104 being attached to one of the user's 102 wrists, the device 104 can also be attached to other arterial sites on the user's body such as one of their forearms, or the upper portion of one of their arms, or their torso, or the upper portion of one of their legs, or the lower portion of one of their legs, or one of their ankles, among other possible arterial sites.

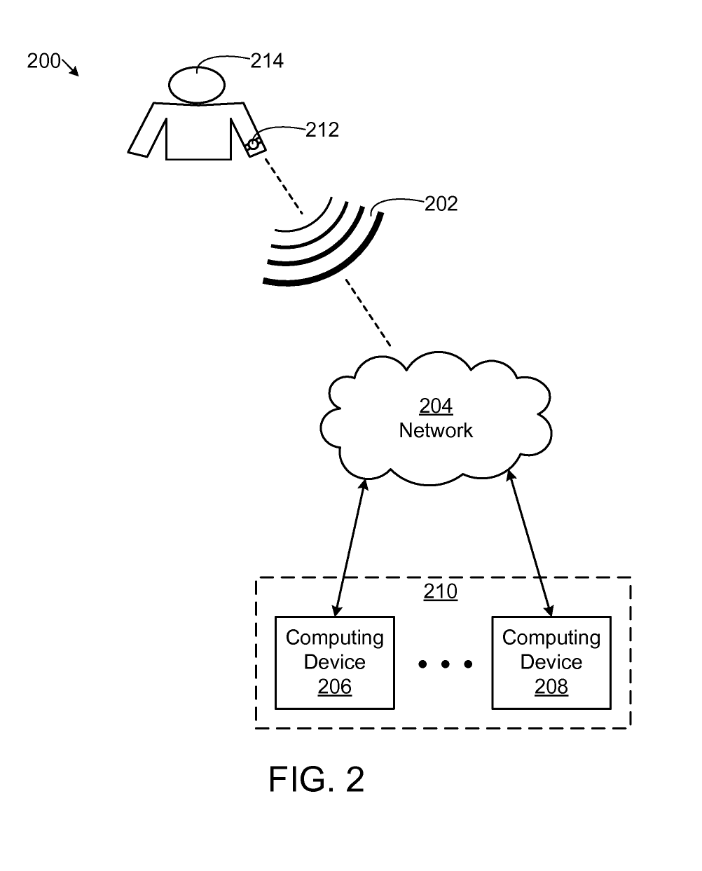

FIG. 2 illustrates another implementation, in simplified form, of an architectural framework for realizing the cardiovascular measurements technique implementations described herein. In the architectural framework 200 exemplified in FIG. 2, a portion of the cardiovascular measurements technique implementations is realized in a portable computing device 212 that is attached to an arterial site on the body of a user 214 as just described (e.g., a wrist of the user in the illustrated implementation). Another portion of the cardiovascular measurements technique implementations is realized in one or more computing devices 206 and 208 that are remotely located from the portable computing device 212. The portable computing device 212 is configured to wirelessly 202 communicate with the remote computing devices 206 and 208 via a data communication network 204 such as the Internet, among other types of networks. The remote computing devices 206 and 208 can also communicate with each other via the network 204. As will be described in more detail hereafter, the portion of the cardiovascular measurements technique implementations that is realized in the remote computing devices 206 and 208 can provide a cloud service 210 to the portable computing device 212 which determines the timing and context for measuring the cardiovascular vital signs of the user 214. The term “cloud service” is used herein to refer to a web application that operates in the cloud, and can be hosted on (e.g., deployed at) a plurality of data centers that can be located in different geographic regions (e.g., different regions of the world), and can be concurrently used by a plurality of remote end users. It is noted that although the architectural framework 200 depicts a single user 214 and a single portable computing device 212, yet another architectural framework implementation (not shown) is also possible where the cloud service is provided to a plurality of portable computing devices each of which is attached to a different user (e.g., the cloud service can determine the timing and context for measuring the cardiovascular vital signs of a plurality of users).

### 1.2 Process Framework

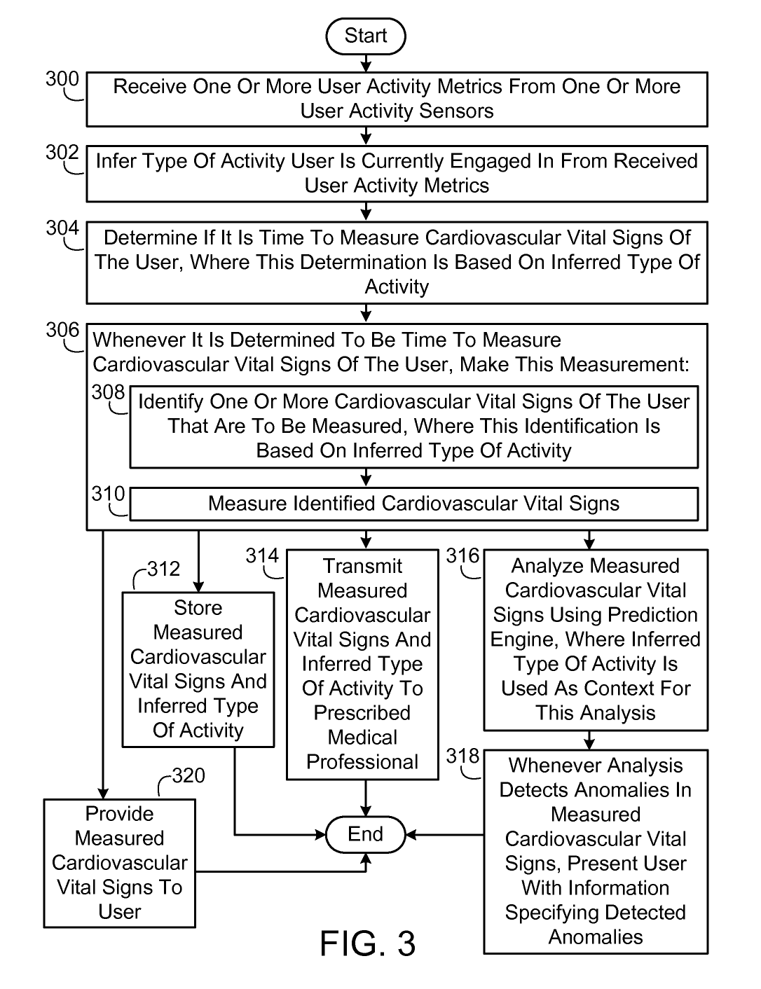

FIG. 3 illustrates one implementation, in simplified form, of a process for measuring the cardiovascular vital signs of a user. As will be appreciated from the more detailed description that follows and referring again to FIG. 1, the process implementation illustrated in FIG. 3 is based on the architectural framework 100 implementation illustrated in FIG. 1 and described heretofore. In other words, in an exemplary implementation the process illustrated in FIG. 3 is realized on the aforementioned portable computing device. As exemplified in FIG. 3, the process starts with receiving one or more user activity metrics from one or more user activity sensors (process action 300). The user activity sensors can include various types of conventional sensing devices that output various types of user activity metrics each of which measures a particular attribute (e.g., a particular property or characteristic) of either the user or their current environment whenever the portable computing device 104 is worn by the user.

More particularly, the user activity sensors can include, but are not limited to, a global positioning system (GPS) receiver, an accelerometer, a gyroscope, a microphone, and a camera. The GPS receiver is attached to the user and is configured to detect the current geographical location (also known as the geolocation) of the user, the current altitude of the user, and the current linear velocity of the user; in other words, the GPS receiver is configured to generate a user geolocation metric, a user altitude metric, and a user velocity metric. The accelerometer is also attached to the user and is configured to detect the current acceleration of the user and the direction thereof; in other words, the accelerometer is configured to generate a user acceleration metric. The gyroscope is also attached to the user and is configured to detect the current physical orientation of the user; in other words, the gyroscope is configured to generate a user physical orientation metric. The microphone is either also attached to the user or in the immediate vicinity of the user, and is configured to capture sound from the user's current environment; in other words, the microphone is configured to generate an environmental sound metric. The camera is either also attached to the user or in the immediate vicinity of the user (e.g., a surveillance camera, or the like), and is configured to capture images of the user's current environment, and to detect the current light-level therein; in other words, the camera is configured to generate an environmental images metric and a light-level metric.

The user activity sensors can also include an altimeter, a wireless communication signal sensor, and a moisture sensor. The altimeter is also attached to the user and is configured to also detect the current altitude of the user; in other words, the altimeter is configured to generate another user altitude metric. The wireless communication signal sensor is also attached to the user and is configured to detect the current strength of a wireless communication signal arriving at the user; in other words, the wireless communication signal sensor is configured to generate a wireless communication signal metric. In one implementation of the cardiovascular measurements technique described herein the wireless communication signal sensor is a cellular signal sensor that is configured to generate a cellular signal strength metric; as is appreciated in the art of wireless communication, the cellular signal strength metric can be used to approximate the current geographical location of the user based on known location of one or more cellular signal transmitters in the vicinity of the user. In another implementation of the cardiovascular measurements technique described herein the wireless communication signal sensor is a Wi-Fi signal sensor that is configured to generate a Wi-Fi signal strength metric; as is also appreciated in the art of wireless communication, the Wi-Fi signal strength metric can be used to approximate the current geographical location of the user based on known location of one or more Wi-Fi signal transmitters in the vicinity of the user. The moisture sensor is also attached to the user and is configured to detect moisture in the user's current environment; in other words, the moisture sensor is configured to generate a moisture metric that can indicate when the user is immersed in water (as would be the case when they are swimming, for example).

The user activity sensors can also include one or more pulse pressure wave (PPW) sensors that are configured into the portable computing device such that whenever the device is attached to an arterial site on the user's body, the PPW sensors are either in close proximity to or in physical contact with the arterial site. The PPW sensors are thus configured to measure a pulse pressure wave as it passes by the arterial site by sensing a change in shape of an artery at the arterial site as the pulse pressure wave passes thereby, regardless of which type of activity the user may be engaged in. A conventional method (such as the photoplethysmography (PPG) method, among other possible methods) can then be used to compute the current heart rate of the user from the measured pulse pressure wave. In other words, the PPW sensors are configured to measure a pulse waveform from which a user heart rate metric can be computed, and whose morphology can be analyzed to derive various other cardiovascular metrics. In one implementation of the cardiovascular measurements technique described herein the PPW sensors are optical-type sensors, each of which is composed of a combination of one or more LED (light emitting diode) illumination sources and one or more light detectors (e.g., photosensors, also known as photodetectors). In another implementation of the cardiovascular measurements technique the PPW sensors are mechanical-type sensors each of which is composed of one or more piezoresistive sensors. Whenever a plurality of PPW sensors is employed, they can all be optical, or they can all be mechanical, or they can be any combination of optical and mechanical. A heart rate variability metric can also be computed from previous heart rate measurements that have been stored as described herein.

Referring again to FIG. 3, after the user activity metrics have been received from the user activity sensors (process action 300), a type of activity the user is currently engaged in (herein sometimes simply referred to as a user activity) is inferred from the received user activity metrics (process action 302). Various methods can be used to make this user activity inference, examples of which are provided hereafter. As will be appreciated from the more detailed description that follows, the cardiovascular measurements technique implementations described herein can infer various types of user activity. The particular number and types of user activity that can be inferred by the cardiovascular measurements technique implementations, and the accuracy of this inference, generally depend on factors such as the particular number and types of user activity metrics that are received, and the particular method that is used to make the inference. The types of user activity that can be inferred by the cardiovascular measurements technique implementations include, but are not limited to, walking in a non-stationary manner (e.g., hiking, or the like), climbing a flight of stairs, riding in a moving vehicle (such as a bus, or a car, or a train, or the like), riding in an airplane, sitting on a stationary object (such as a chair, or a bench, or the like), sleeping, lying down but not sleeping, and performing various types of physical exercise (such as jogging in a non-stationary manner (e.g., running on the open road, or the like), jogging on a treadmill, walking on a treadmill, biking, playing tennis, golfing, and swimming, among many other types of physical exercise).

It is noted that the just-described user activities are exemplary, and many additional types of user activity may also be inferred in accordance with the cardiovascular measurements technique implementations described herein. It is also noted that the cardiovascular measurements technique implementations can employ any combination of the aforementioned different types of user activity sensors. As will be appreciated from the more detailed description that follows, certain combinations of user activity sensors may output a set of user activity metrics that overlap with each other to some degree and thus may provide for an even more robust (e.g., more accurate) inference of the type of activity the user is currently engaged in.

Referring again to FIG. 3, in an exemplary implementation of the cardiovascular measurements technique described herein the user activity inference of action 302 is made using activity classifier logic. This particular implementation will hereafter be referred to as the classifier logic implementation of the cardiovascular measurements technique. In one version of the classifier logic implementation of the cardiovascular measurements technique the activity classifier logic includes a set of pre-configured user activity profiles (hereafter sometimes simply referred to as activity profiles). Each of the activity profiles in the set thereof corresponds to a different type of user activity, and specifies either a value or a range of values for each of the user activity metrics that are expected to be received whenever the user is engaged in this different type of user activity. The user activity inference of action 302 is made by comparing the user activity metrics received in action 300 to the set of pre-configured activity profiles, identifying one of the activity profiles in this set whose user activity metrics most closely match the user activity metrics received in action 300, and assigning the type of user activity that corresponds to the identified activity profile to be the inferred type of activity.

Each of the activity profiles in the set of pre-configured activity profiles can be pre-configured using various methods. For example, in one implementation of the cardiovascular measurements technique each of the activity profiles in the set thereof can be pre-configured by receiving one or more user activity metrics while a given user performs the type of user activity that corresponds to the activity profile. Each of the received user activity metrics can then be processed to generate either a value or a range of values corresponding thereto. Each of the received user activity metrics and the value or a range of values corresponding thereto can then be stored in the activity profile. In another implementation of the cardiovascular measurements technique each of the activity profiles in the set thereof can be automatically generated using one or more conventional statistical correlation methods. A combination of these two implementations can also be used (e.g., a user can manually tune each of the automatically generated activity profiles).

In another version of the classifier logic implementation of the cardiovascular measurements technique described herein the activity classifier logic includes a conventional classifier (such as an artificial neural network, or a Bayesian statistical classifier, among other types of conventional classifiers) that is trained using a training data set such as the aforementioned set of pre-configured activity profiles.

Referring again to FIG. 3, the accuracy of the user activity inference of action 302 can optionally be sharpened/enhanced using various methods. By way of example but not limitation, in yet another version of the classifier logic implementation of the cardiovascular measurements technique described herein each of the activity profiles in the set of pre-configured activity profiles can optionally include significance metrics that specify the significance of each of the user activity metrics in the activity profile to the type of user activity that corresponds to the activity profile. In other words, a significance metric can optionally be assigned to each of the user activity metrics that is stored in each of the activity profiles, where this significance metric specifies the significance of the stored user activity metric to the inference of the type of user activity that corresponds to the activity profile. For example and as will be appreciated from the more detailed description that follows, in a situation where the user activity sensors include an accelerometer and a GPS receiver, the aforementioned riding in a moving vehicle user activity may place a higher significance on the user velocity metric that is received from the GPS receiver than the user acceleration metric that is received from the accelerometer. During the aforementioned action of comparing the user activity metrics received in action 300 to the set of pre-configured activity profiles, the activity classifier logic can weight each of the received user activity metrics according to the significance metrics that are assigned to each of the user activity metrics in each of the activity profiles.

In yet another version of the classifier logic implementation of the cardiovascular measurements technique described herein, the activity classifier logic can optionally compute a confidence metric that specifies the level of confidence the activity classifier logic has in its inference of a type of activity from the received user activity metrics. Whenever this confidence metric is less than a prescribed confidence level threshold (e.g., 90 percent confidence, which can be considered to represent an accurate inference, among other possible confidence thresholds), the cardiovascular measurements technique may then prompt (e.g., ask) the user to specify the type of user activity that generated the received user activity metrics. This user prompt and activity specification can be realized in various ways. For example, in the case where the portable computing device includes a touch-enabled display subsystem the prompt can be displayed to the user and can include a set of possible activities from which the user may manually make a selection; the user may also specify an activity that is not in the set of possible activities. In the case where the portable computing device includes sound output and voice recognition functionality the prompt can be audibly output from the portable computing device, and the user may verbally specify the type of user activity that generated the received user activity metrics.

It will be appreciated that a low confidence metric can result from a variety of circumstances, such as when the user performs a given type of activity in a peculiar or user-specific manner, which can result in user activity metrics being generated that substantially differ from those that are stored in the activity profile corresponding to this type of user activity, or in the training data set used to train the classifier. Upon receiving the user's manual identification of the type of user activity that resulted in the received user activity metrics, the cardiovascular measurements technique may then adjust the activity classifier logic so as to be able to accurately infer this type of user activity the next time similar user activity metrics are received (e.g., the activity profile corresponding to this type of user activity may be adjusted, or the training data set may be adjusted and the classifier may be retrained).

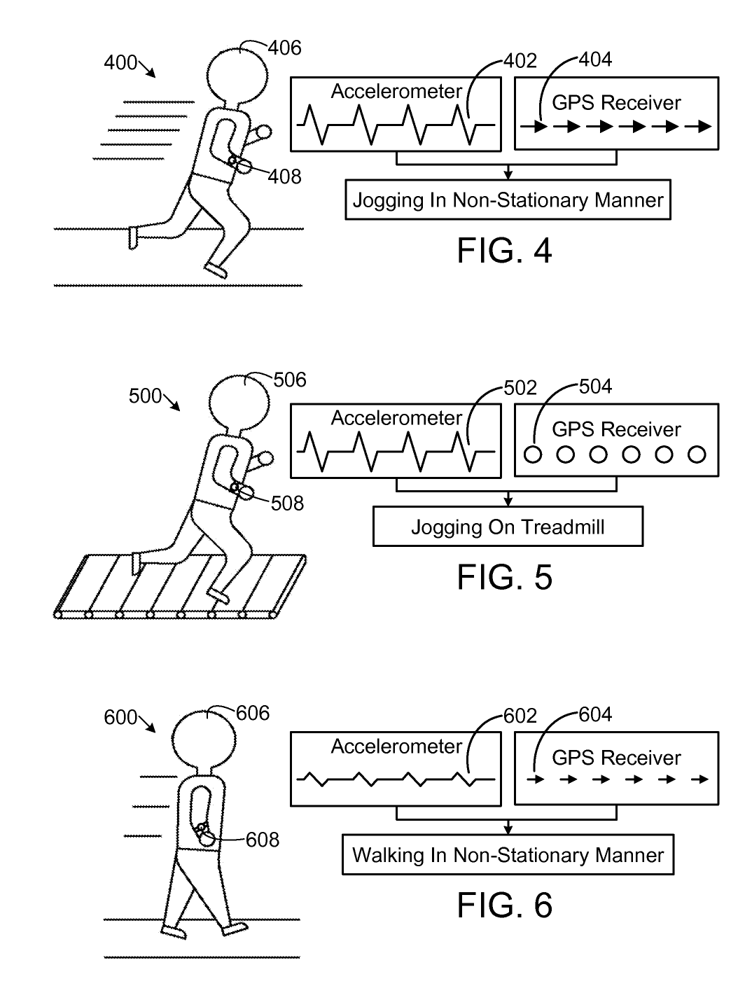

FIG. 4 illustrates one scenario, in simplified form, of one exemplary type of activity the user is currently engaged in being inferred from user activity metrics that are received from an accelerometer and a GPS receiver. As exemplified in FIG. 4, the user 406 is jogging in the aforementioned non-stationary manner 400 and has the portable computing device 408 attached to one of their wrists. While the jogging in a non-stationary manner activity 400 is occurring, the user acceleration metric 402 generated by the accelerometer indicates a modest repeating impulse that is caused by the user's 406 footsteps while they are performing this activity 400, and the user velocity metric 404 generated by the GPS receiver indicates a low-velocity directional movement having a speed (e.g., approximately six kilometers per hour) that is within a range of speeds that is commonly associated with this activity 400. Accordingly, the cardiovascular measurements technique implementations described herein can infer from these user activity metrics 402 and 404 (as well as any other user activity metrics that might be received) that the user 406 is jogging outdoors 400.

FIG. 5 illustrates another scenario, in simplified form, of another exemplary type of activity the user is currently engaged in being inferred from user activity metrics that are received from the accelerometer and the GPS receiver. As exemplified in FIG. 5, the user 506 is jogging on a treadmill 500 and has the portable computing device 508 attached to one of their wrists. While the jogging on a treadmill activity 500 is occurring, the user acceleration metric 502 generated by the accelerometer indicates a modest repeating impulse that is caused by the user's 506 footsteps while they are performing this activity 500, and the user velocity metric 504 generated by the GPS receiver indicates that the user 506 is substantially stationary. Accordingly, the cardiovascular measurements technique implementations described herein can infer from these user activity metrics 502 and 504 (as well as any other user activity metrics that might be received) that the user 506 is jogging on a treadmill 500.

FIG. 6 illustrates yet another scenario, in simplified form, of yet another exemplary type of activity the user is currently engaged in being inferred from user activity metrics that are received from the accelerometer and the GPS receiver. As exemplified in FIG. 6, the user 606 is walking in the aforementioned non-stationary manner 600 and has the portable computing device 608 attached to one of their wrists. While the walking in a non-stationary manner activity 600 is occurring, the user acceleration metric 602 generated by the accelerometer indicates a repeating impulse that is caused by the user's 606 footsteps while they are performing this activity 600, where this repeating impulse has a lower magnitude than that which is generated during the aforementioned jogging activities. The user velocity metric 604 generated by the GPS receiver indicates very-low-velocity directional movement having a speed (e.g., approximately four kilometers per hour) that is within a range of speeds that is commonly associated with the walking in a non-stationary manner activity 600. Accordingly, the cardiovascular measurements technique implementations described herein can infer from these user activity metrics 602 and 604 (as well as any other user activity metrics that might be received) that the user 606 is walking in a non-stationary manner 600.

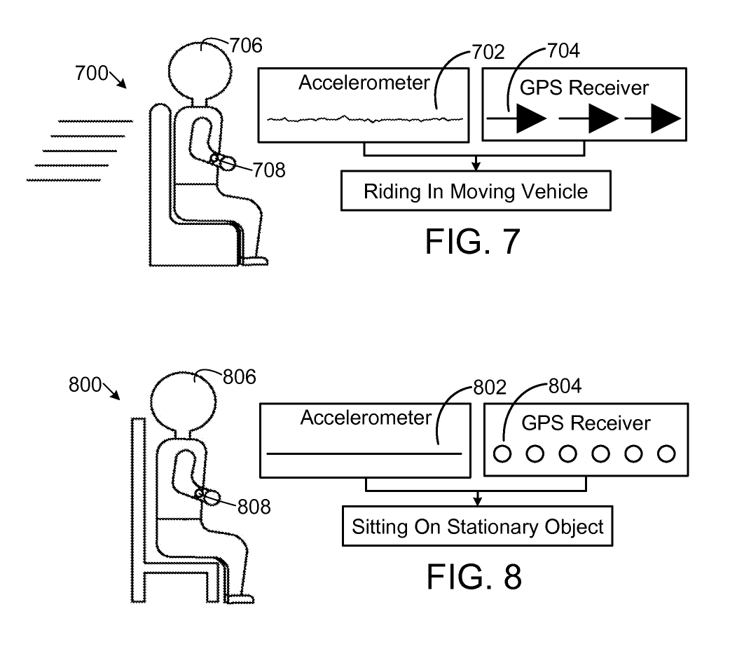

FIG. 7 illustrates yet another scenario, in simplified form, of yet another exemplary type of activity the user is currently engaged in being inferred from user activity metrics that are received from the accelerometer and the GPS receiver. As exemplified in FIG. 7, the user 706 is riding in a moving vehicle 700 and has the portable computing device 708 attached to one of their wrists. While the riding in a moving vehicle activity 700 is occurring, the user acceleration metric 702 generated by the accelerometer indicates a latent vibration that may be caused by uneven road surfaces (among other factors) while the user 706 is engaged in this activity 700, and the user velocity metric 704 generated by the GPS receiver indicates a moderate to high-velocity directional movement having a speed that is within a range of speeds that is commonly associated with a moving vehicle. Accordingly, the cardiovascular measurements technique implementations described herein can infer from these user activity metrics 702 and 704 (as well as any other user activity metrics that might be received) that the user 706 is riding in a moving vehicle 700.

FIG. 8 illustrates yet another scenario, in simplified form, of yet another exemplary type of activity the user is currently engaged in being inferred from user activity metrics that are received from the accelerometer and the GPS receiver, along with a gyroscope (not shown). As exemplified in FIG. 8, the user 806 is sitting on a stationary object 800 and has the portable computing device 808 attached to one of their wrists. While the sitting on a stationary object activity 800 is occurring, both the user acceleration metric 802 generated by the accelerometer and the user velocity metric 804 generated by the GPS receiver indicate that the user 806 is substantially stationary. The user physical orientation metric (not shown) generated by the gyroscope indicates that the user 806 is in an upright position. Accordingly, the cardiovascular measurements technique implementations described herein can infer from the user acceleration and user velocity metrics 802 and 804, and the user physical orientation metric (as well as any other user activity metrics that might be received) that the user 806 is sitting on a stationary object activity 800.

Referring again to FIG. 3, after a user activity has been inferred from the received user activity metrics (process action 302), a determination is made as to if it is time to measure the cardiovascular vital signs of the user, where this determination is based on the inferred type of activity (process action 304). It will be appreciated that process actions 302 and 304 advantageously reduce the processing load, conserve battery power, and conserve data storage space on the portable computing device by eliminating the need to continuously measure the cardiovascular vital signs of the user. Whenever it is determined to be time to measure the cardiovascular vital signs of the user, this measurement is made (process action 306) as follows. One or more of the cardiovascular vital signs of the user that are to be measured are identified, where this identification is also based on the inferred type of activity (process action 308). The identified cardiovascular vital signs are then measured (process action 310). As will be appreciated from the more detailed description that follows, in certain implementations of the cardiovascular measurements technique described herein, the user may need to perform a specific act that initiates the measurement of certain ones of their cardiovascular vital signs. An example of such an act that initiates a pulse transit time measurement is provided hereafter. In such implementations, the user is prompted to perform the specific act, where this prompting can be realized in various ways. For example, in the case where the portable computing device includes a display subsystem the prompt to perform the specific act can be displayed to the user. In the case where the portable computing device includes sound output functionality, the prompt to perform the specific act can be audibly output from the portable computing device. In the case where the portable computing device includes a vibration motor, the user can be prompted by turning the vibration motor on, thus causing the portable computing device to vibrate which will be felt by the user.

Referring again to FIG. 3, in an exemplary implementation of the cardiovascular measurements technique described herein the determination of action 304 and the identification of action 308 are made using a pre-configured set of logic. For each type of user activity that can be inferred from the received user activity metrics, this set of logic specifies if the cardiovascular vital signs of the user are to measured; in the case where the set of logic specifies that the cardiovascular vital signs of the user are to measured, the set of logic also identifies which one or more cardiovascular vital signs of the user are to be measured and specifies when this measurement is to be made. By way of example but not limitation, for a given type of user activity that can be inferred from the received user activity metrics this set of logic may specify that one or more of the identified cardiovascular vital signs are to be measured either while the inferred type of activity is occurring, or immediately after the inferred type of activity has completed, or one or more prescribed periods of time after the inferred type of activity has completed, or any combination thereof.

As described heretofore, the cardiovascular measurements technique implementations described herein can measure various cardiovascular vital signs including, but not limited to, a pulse transit time, a pulse wave velocity, a heart rate, heart rate variability, a pulse waveform whose morphology can be analyzed to derive various other cardiovascular metrics, blood pressure, blood pressure variability, and any combination thereof. Exemplary methods for measuring these cardiovascular vital signs are described in more detail hereafter.

Referring again to FIG. 3, after the cardiovascular vital signs of the user have been measured (process action 306), various things can be done with the measurements, examples of which include, but are not limited to, the following. The measured cardiovascular vital signs of the user and the inferred type of activity can be stored (process action 312), where the inferred type of activity serves as a context for the measured cardiovascular vital signs. The stored data may then be provided to a clinical research database that researches can use to identify correlations between certain types of user activity and the cardiovascular vital signs of users. The measured cardiovascular vital signs of the user and the inferred type of activity can also be transmitted to a prescribed medical professional (process action 314) such as a cardiologist (among other types of medical professionals). The medical professional may interpret the measured cardiovascular vital signs in the context of the inferred type of activity, and based on this interpretation may diagnose any cardiovascular diseases that the user may have and recommend a plan of treatment to the user. The measured cardiovascular vital signs of the user can also be analyzed using a prediction engine, where the inferred type of activity is used as a context for this analysis (process action 316), and whenever this analysis detects one or more anomalies in the measured cardiovascular vital signs of the user, the user can then be presented with information specifying the detected anomalies (process action 318), where this information may include recommended next steps to be taken by the user. It will be appreciated that this detected anomalies information can be presented to the user in various ways. For example, in the case where the portable computing device includes a touch-enabled display subsystem the detected anomalies information can be displayed to the user. In the case where the portable computing device includes sound output functionality the detected anomalies information can be audibly output from the portable computing device. One or more of the measured cardiovascular vital signs can also be provided (e.g., displayed or audibly output) to the user (process action 320).

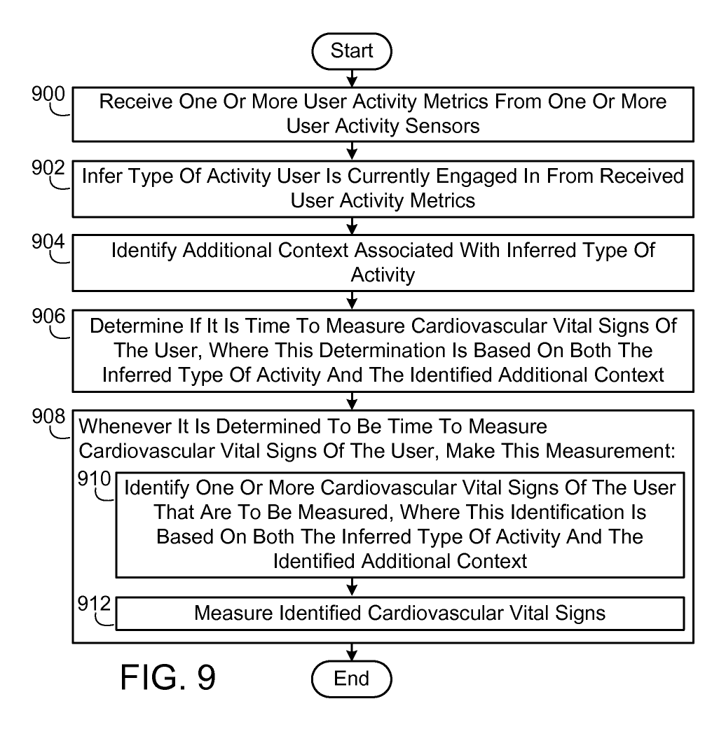

FIG. 9 illustrates another implementation, in simplified form, of a process for measuring the cardiovascular vital signs of a user. As will be appreciated from the more detailed description that follows and referring again to FIG. 1, the process illustrated in FIG. 9 is based on the architectural framework 100 illustrated in FIG. 1 and described heretofore. In other words, in an exemplary implementation the process illustrated in FIG. 9 is realized on the portable computing device. As exemplified in FIG. 9, the process starts with receiving one or more user activity metrics from a one or more user activity sensors (process action 900), where the user activity sensors can include any of the types of sensing devices described heretofore. A type of activity the user is currently engaged in is then inferred from the received user activity metrics (process action 902), where this user activity inference can be made using any of the methods described heretofore. Additional context that is associated with the inferred type of activity is then identified (process action 904). Exemplary methods for identifying this additional context and exemplary types of additional context that can be identified are described in more detail hereafter. A determination is then made as to if it is time to measure the cardiovascular vital signs of the user, where this determination is based on both the inferred type of activity and the identified additional context (process action 906). It will be appreciated that process actions 902, 904 and 906 advantageously reduce the processing load, conserve battery power, and conserve data storage space on the portable computing device by eliminating the need to continuously measure the cardiovascular vital signs of the user. Whenever it is determined to be time to measure the cardiovascular vital signs of the user, this measurement is made (process action 908) as follows. One or more of the cardiovascular vital signs of the user that are to be measured are identified, where this identification is also based on both the inferred type of activity and the identified additional context (process action 910). The identified cardiovascular vital signs are then measured (process action 912). In an exemplary implementation of the cardiovascular measurements technique described herein the determination of action 906 and the identification of action 910 are made using a modified version of the aforementioned pre-configured set of logic that also takes into account various types of additional context that may be associated with each type of user activity that can be inferred from the received user activity metrics.

Referring again to FIG. 9, the additional context identification of action 904 can be implemented in various ways. For example, in one implementation of the cardiovascular measurements technique described herein an online calendar for the user (such as the conventional Google Calendar, among many other types of conventional online calendars) may be accessed in order to identify additional context that is associated with a given inferred type of activity (e.g., in a situation where it is inferred that the user is currently sitting on a stationary object, an online calendar for the user might indicate that the user is currently attending a meeting with their divorce attorney). In another implementation of the cardiovascular measurements technique various “digital activities” of the user (e.g., the user's email messages, or their text messages, or their web search activities, or the like) may be analyzed in order to identify additional context that is associated with a given inferred type of activity. In yet another implementation of the cardiovascular measurements technique images from a surveillance camera that is in the immediate vicinity of the user may be accessed in order to identify additional context that is associated with a given inferred type of activity (e.g., in a situation where it is inferred that the user is currently sitting on a stationary object, images from a surveillance camera might indicate that the user is currently drinking a cup of coffee in a coffee shop, or might indicate that the user is currently watching television).

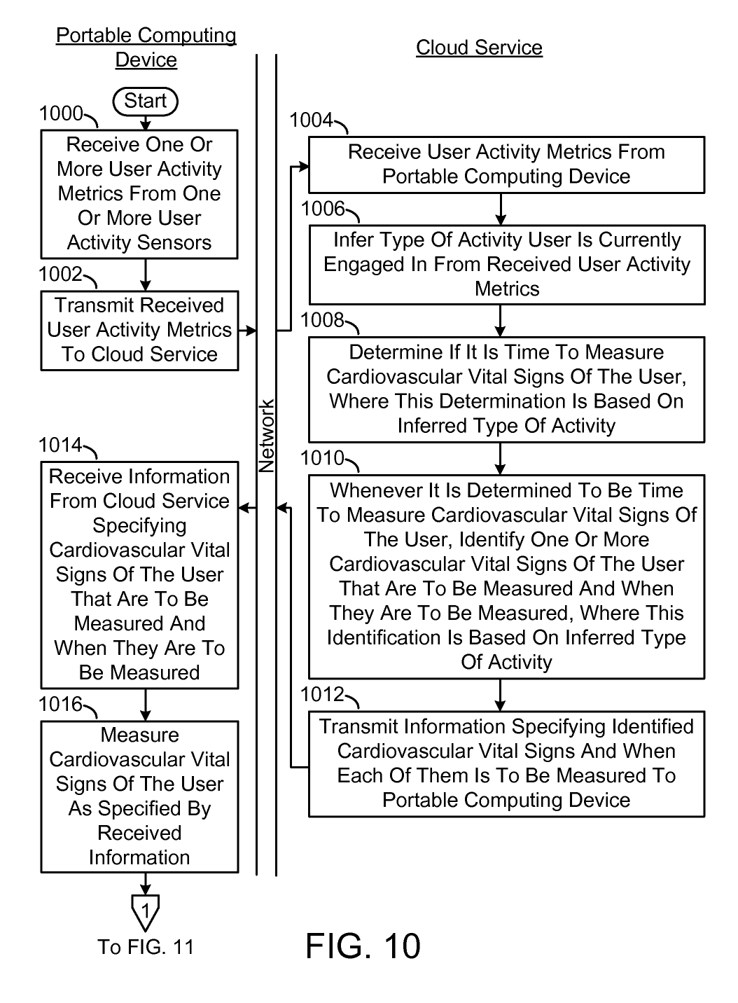

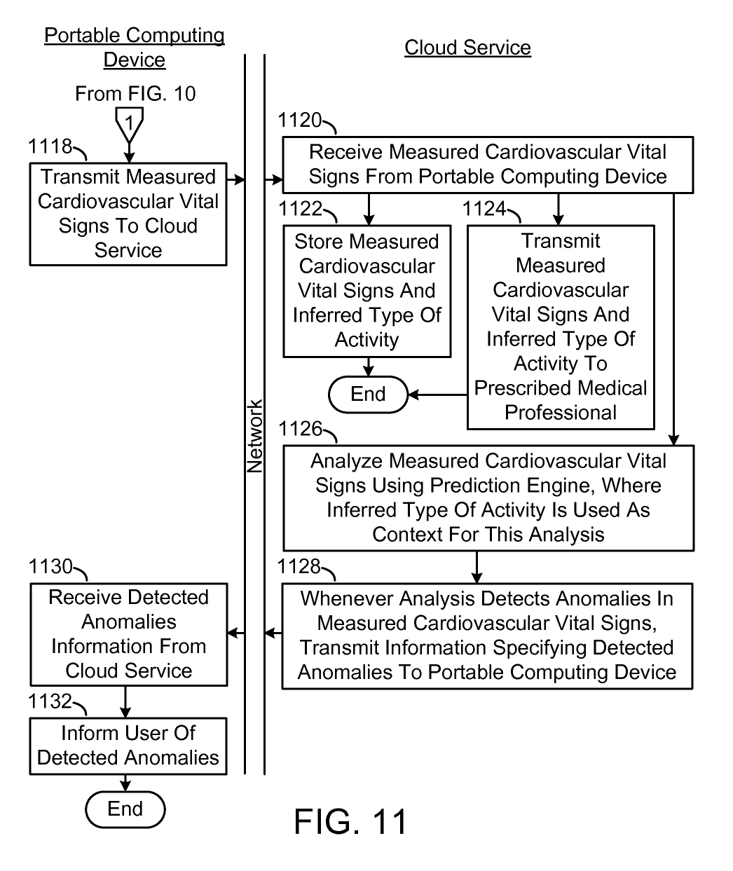

FIGS. 10 and 11 illustrate yet another implementation, in simplified form, of a process for measuring the cardiovascular vital signs of a user. As will be appreciated from the more detailed description that follows and referring again to FIG. 2, the process illustrated in FIGS. 10 and 11 is based on the architectural framework 200 implementation illustrated in FIG. 2 and described heretofore. In other words, in an exemplary implementation one portion of the process illustrated in FIGS. 10 and 11 is realized on the portable computing device, and another portion of this process is realized on the aforementioned computing devices that are remotely located from the portable computing device and provide the cloud service to the portable computing device. More particularly, the process illustrated in FIGS. 10 and 11 corresponds to an exemplary implementation of the aforementioned cloud service 210 that determines the timing and context for measuring the cardiovascular vital signs of the user 214 who is attached to the portable computing device 212 as described herein. As exemplified in FIG. 10, the process starts with the portable computing device receiving one or more user activity metrics from one or more user activity sensors (process action 1000), where the user activity sensors can include any of the types of sensing devices described heretofore. The portable computing device then transmits the received user activity metrics to the cloud service (process action 1002).

Referring again to FIG. 10, upon receiving the one or more user activity metrics transmitted from the portable computing device (process action 1004), the cloud service infers a type of activity the user is currently engaged in from the received user activity metrics (process action 1006), where this user activity inference can be made using any of the methods described heretofore. The cloud service then determines if it is time to measure the cardiovascular vital signs of the user, where this determination is based on the inferred type of activity (process action 1008). It will be appreciated that process actions 1006 and 1008 advantageously reduce the processing load and conserve battery power on the portable computing device, and conserve data storage space on the computing devices that are remotely located from the portable computing device, by eliminating the need to continuously measure the cardiovascular vital signs of the user. Whenever it is determined to be time to measure the cardiovascular vital signs of the user, the cloud service then identifies one or more of the cardiovascular vital signs of the user that are to be measured and when they are to be measured, where this identification is also based on the inferred type of activity (process action 1010). Exemplary times that a given cardiovascular vital sign can be measured have been described heretofore. The cloud service then transmits information specifying the cardiovascular vital signs of the user that are to be measured and when they are to be measured to the portable computing device (process action 1012). The determination of action 1008 and the identification of action 1010 can be made using the pre-configured set of logic as described heretofore.

Referring again to FIG. 10 and as exemplified in FIG. 11, upon receiving the information transmitted from the cloud service specifying the cardiovascular vital signs of the user that are to be measured and when they are to be measured (process action 1014), the portable computing device measures the cardiovascular vital signs of the user as specified by the received information (process action 1016). The portable computing device then transmits the measured cardiovascular vital signs of the user to the cloud service (process action 1118). Upon receiving the measured cardiovascular vital signs of the user transmitted from the portable computing device (process action 1120), the cloud service can do various things, examples of which include, but are not limited to, the following. The measured cardiovascular vital signs of the user and the inferred type of activity can be stored (process action 1122) as described heretofore. The measured cardiovascular vital signs of the user and the inferred type of activity can also be transmitted to a prescribed medical professional (process action 1124) as described heretofore. The measured cardiovascular vital signs of the user can also be analyzed using a prediction engine, where the inferred type of activity is used as a context for this analysis (process action 1126), and whenever this analysis detects one or more anomalies in the measured cardiovascular vital signs of the user, information specifying the detected anomalies can be transmitted to the portable computing device (process action 1128), where this information may include recommended next steps to be taken by the user. Upon receiving this detected anomalies information transmitted from the cloud service (process action 1130), the portable computing device can inform the user of the detected anomalies (process action 1132) (e.g., the portable computing device may either display or audibly communicate the detected anomalies to the user, among other possible methods for communicating information to the user).

### 1.3 Measuring Pulse Transit Time

This section provides a description, in simplified form, of an exemplary method that can be used to measure a pulse transit time. The pulse transit time measurement method described in this section (hereafter sometimes simply referred to as the PTT measurement method) assumes that the primary ECG electrodes are configured into the portable computing device such that whenever the device is attached to an arterial site on a user's body, the primary ECG electrodes are in electrical contact with (e.g., touch) the arterial site. Accordingly, the term “primary” is employed just to indicate that, whenever the portable computing device is attached to an arterial site on a user's body the primary ECG electrodes maintain electrical contact with the arterial site regardless of which type of activity the user may be engaged in. In one version of the PTT measurement method the primary ECG electrodes are of a rigid, metallic type. In another version of the PTT measurement method the primary ECG electrodes are of a flexible, conductive fabric type. Whenever a plurality of primary ECG electrodes is employed, they can all be of the same type, or they can be any combination of the foregoing types.

The pulse transit time measurement method described in this section also assumes that the secondary ECG electrodes are configured into the portable computing device such that whenever the device is attached to the arterial site on the user's body, the secondary ECG electrodes are electrically isolated from the arterial site. In addition, the secondary ECG electrodes are positioned on the portable computing device so as to allow these electrodes to be contacted by a second location on the user's body that is located on the opposite side of the user's heart from the arterial site. As will be described in more detail hereafter, this contact with the second location on the user's body takes place whenever a pulse transit time measurement is to be made. Accordingly, the term “secondary” is employed just to indicate that, whenever the portable computing device is attached to the arterial site on the user's body the secondary ECG electrodes do not touch the arterial site. As with the primary ECG electrodes, in one version of the PTT measurement method the secondary ECG electrodes are of a rigid, metallic type. In another version of the PTT measurement method the secondary ECG electrodes are of a flexible, conductive fabric type. Whenever a plurality of secondary ECG electrodes is employed, they can all be of the same type, or they can be any combination of the foregoing types.

It is noted that whenever a plurality of electrodes is employed for the primary or secondary ECG electrodes, they can be configured in different ways. In one version of the pulse transit time measurement method described in this section the plurality of electrodes associated with a given ECG electrode set (either primary, or secondary, or both) is electrically connected. This version is useful in assuring that at least one of the electrodes in the ECG electrode set makes electrical contact with the user's body. In another version of the PTT measurement method the plurality of electrodes associated with a given ECG electrode set (either primary, or secondary, or both) is electrically isolated. This version is useful in allowing the output from the electrically isolated electrode exhibiting the highest quality signal to be chosen. In yet another version of the PTT measurement method the plurality of electrodes associated with a given ECG electrode set (either primary or secondary, but not both) includes a grounded electrode. This version is useful since some signal processing schemes use a grounded electrode configuration.

As indicated previously, pulse transit time is the amount of time it takes for a pulse pressure wave to travel through an artery (or series of arteries) from one arterial site on the user's body to another arterial site on the user's body. It is noted that this is not the time it takes for any given molecule of blood to move the same distance. Rather, this is the time it takes for the pulse pressure wave to travel through the user's blood (which is very fast—on the order of 200-400 milliseconds from a typical user's heart to wrist). In the context of the cardiovascular measurements technique implementations described herein, pulse transit time refers to the amount of time it takes for the pulse pressure wave to travel from the user's heart, through an artery (or series of arteries), to the arterial site on the user's body to which the portable computing device is attached (e.g., the wrist of the user). Advantageously, pulse transit time correlates with other cardiovascular metrics such as blood pressure, arterial compliance, and the hardening of artery walls.

The primary and secondary ECG electrodes are configured to measure the aforementioned ECG signal whenever the secondary ECG electrodes make electrical contact with the second location on the user's body. The ECG signal measurement is used to identify the time when a pulse pressure wave leaves the user's heart, where this identification is performed using conventional signal processing methods (which include noise reduction and motion compensation). The PPW sensors are configured to measure a pulse waveform which is used to identify the time when the pulse pressure wave arrives at the arterial site on the user's body from the user's heart. A pulse transit time between the user's heart and the arterial site on the user's body is then computed by subtracting the time when the pulse pressure wave leaves the user's heart from the time when the pulse pressure wave arrives at the arterial site on the user's body. The pulse transit time is thus the amount of time that passes between when the pulse pressure wave leaves the user's heart and when the pulse pressure wave arrives at the arterial site on the user's body.

The pulse transit time measurement can be initiated in many different ways, examples of which will now be provided. In an exemplary implementation of the cardiovascular measurements technique described herein where the user has the portable computing device attached to their left wrist, the pulse transit time measurement is initiated whenever the user performs the act of touching the secondary ECG electrodes on this device with or to the second location on the user's body. For example, the user may touch the secondary ECG electrodes with one or more fingers of their right hand (which is on the opposite side of the user's heart from their left wrist). The user may also reach across their body with their left wrist and touch the secondary ECG electrodes to a location on their body that is on the opposite side of the user's heart from their left wrist (such as the right side of their torso, among other possible locations).

The pulse transit time measurement method described in this section can be realized using one or more computing devices. For example, the PTT measurement method can be fully realized in the portable computing device. Alternatively, one portion of the PTT measurement method can be realized in the portable computing device, and another portion of the PTT measurement method can be realized in the aforementioned one or more remote computing devices that provide a cloud service to the portable computing device. For example, after the pulse transit time measurement is initiated as just-described, the portable computing device can receive the ECG signal measurement from the primary and secondary ECG electrodes, and can receive the pulse waveform measurement from the PPW sensors, and can then send the received ECG signal measurement and pulse waveform measurement to the cloud service. Upon receiving the ECG signal measurement and pulse waveform measurement from the portable computing device, the cloud service can then perform the aforementioned signal processing and computation associated with measuring pulse transit time.

### 1.4 Measuring Pulse Wave Velocity

This section provides a description, in simplified form, of an exemplary method that can be used to measure a pulse wave velocity. It is noted that while a pulse transit time measurement is useful and has some correlation to a user's cardiovascular health, pulse transit time by itself does not tell the full cardiovascular health picture. The distance a pulse pressure wave travels between the user's heart and the aforementioned arterial site on the user's body has significance. For example, as indicated previously, the speed at which a pulse pressure wave travels through an artery (or series of arteries) is indicative of arterial stiffness. In general, the faster the pulse pressure wave travels, the stiffer the artery (or arteries) the wave travels through tends to be. Stiffer arteries are generally counter-indicative of good cardiovascular health. Thus, two users could have the same measured pulse transit time but if the distance the pulse pressure wave traveled is significantly different for these two users, one user might be in good cardiovascular health and the other user might not be in good cardiovascular health. For example, if the pulse transit time is measured from the heart to the wrist of both a tall user having stiffer arteries and a short user having more flexible arteries, because the arterial distance from the heart to the wrist of the tall user is larger than the arterial distance from the heart to the wrist of the short user, these two users could have the same measured pulse transit time even though the pulse pressure wave travels faster through the arteries of the tall user (thus indicating that the tall user has stiffer arteries and possible poorer cardiovascular health than the short user). Thus, measuring the speed of the pulse pressure wave along with the pulse transit time is advantageous since the combination of these two measurements could be more indicative of cardiovascular health than just measuring the pulse transit time.

As indicated previously, pulse wave velocity is the average speed at which a pulse pressure wave travels through an artery (or a series of arteries) from one arterial site on the user's body to another arterial site on the user's body. In the context of the cardiovascular measurements technique implementations described herein, pulse wave velocity refers to the average speed of the pulse pressure wave as it travels from the user's heart, through an artery (or series of arteries), to the arterial site on the user's body to which the portable computing device is attached (e.g., the wrist of the user). After the pulse transit time has been measured, an estimate of the arterial travel distance between the user's heart and the arterial site on the user's body (which is hereafter sometimes simply referred to as the arterial travel distance) is obtained. The pulse wave velocity is then computed by dividing the arterial travel distance by the measured pulse transit time. The just-described estimate of the arterial travel distance can be obtained using various methods, some examples of which will now be provided.

For applications of the cardiovascular measurements technique implementations described herein that take place in either a research facility or a clinical setting, the arterial travel distance can be measured directly via conventional x-ray or catheter insertion methods. A medical technician can also mark locations on the user's body that roughly correspond to the likely location(s) of the artery or series of arteries through which the pulse pressure wave travels, and then use a tape measure to estimate the arterial travel distance.

For applications of the cardiovascular measurements technique implementations described herein that take place outside of a research facility or clinical setting (such as when the user is going about their normal day), a camera-based system can be used to capture images (e.g., both color images and depth images, among other types and combinations of images) of the user, and then conventional methods can be used to estimate the arterial travel distance from the captured images. A numerical model can also be generated that estimates arterial travel distance for a given user based on various prescribed body metrics. More particularly, a database can be created that includes direct arterial travel distance measurements between the heart and various prescribed arterial sites (e.g., the wrist and the ankle, among others) for a variety of users. For each of the users whose direct arterial travel distance measurements is included in the database, demographic data for the user can also be collected and added to the database. Examples of such demographic data can include one or more body metrics for the user such as their height, their weight, their age, their gender, and their race, among other body metrics. Appropriate conventional methods can then be used to learn a numerical model that correlates these body metrics to an arterial travel distance between the heart and the various prescribed arterial sites. Once the numerical model is available, the body metrics for a new user (whose arterial travel distances were not directly measured) can be collected, and the model can be used to estimate the arterial travel distance between the new user's heart and the arterial site of interest on the their body (assuming of course that the arterial site of interest is one of the sites that was used to generate the model).

### 1.5 Measuring Blood Pressure

As is appreciated in the arts of medicine and cardiovascular health, the user's blood pressure can be measured using various conventional methods. In an exemplary implementation of the cardiovascular measurements technique described herein conventional methods are used to compute the current blood pressure of the user from the measured pulse transit time, where this computation is based on the aforementioned known correlation between pulse transit time and blood pressure. A blood pressure variability metric can also be computed from previous blood pressure measurements that have been stored as described herein.

### 1.6 System Framework

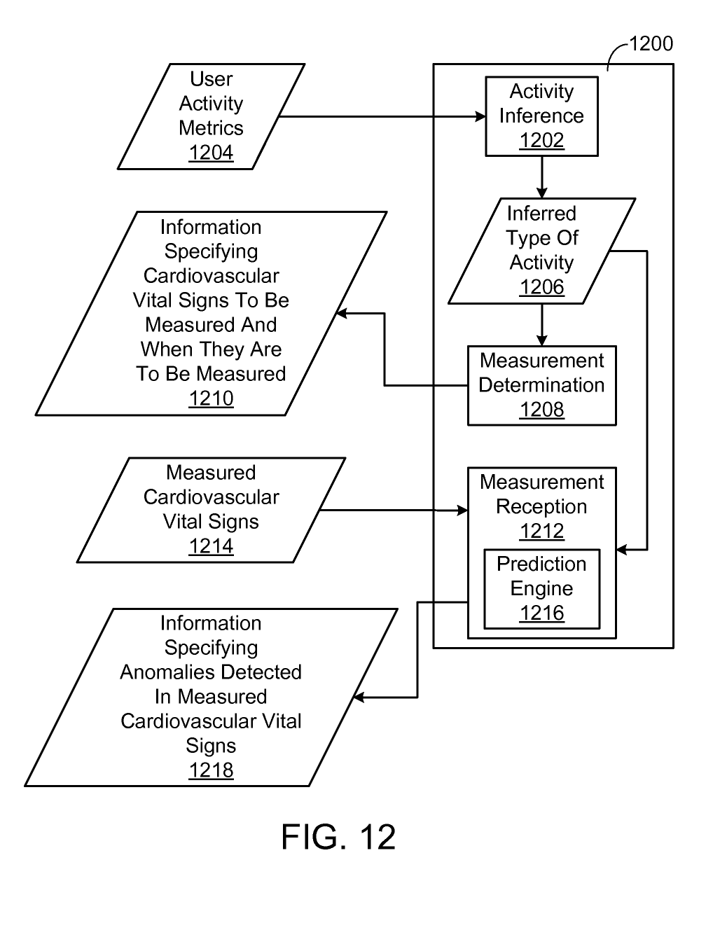

FIG. 12 illustrates an exemplary implementation, in simplified form, of a system framework for measuring the cardiovascular vital signs of a user. As exemplified in FIG. 12, the system framework 1200 includes an activity inference module 1202, a measurement determination module 1208, and a measurement reception module 1212. The activity inference module 1202 receives one or more user activity metrics 1204 and infers a type of activity the user is currently engaged in 1206 from these metrics 1204. The measurement determination module 1208 uses the inferred type of activity 1206 to determine if it is time to measure the cardiovascular vital signs of the user. Whenever the measurement determination module 1208 determines that it is time to measure the cardiovascular vital signs of the user, the measurement determination module 1208 uses the inferred type of activity 1206 to identify one or more of the cardiovascular vital signs of the user that are to be measured and when they are to be measured. The measurement determination module 1208 then outputs information specifying the cardiovascular vital signs of the user that are to be measured and when they are to be measured 1210.

Referring again to FIG. 12, the measurement reception module 1212 receives the measured cardiovascular vital signs of the user 1214 and then can do a variety of things such as one or more of the following. The measurement reception module 1212 can store the measured cardiovascular vital signs of the user 1214 and the inferred type of activity 1206. The measurement reception module 1212 can also transmit the measured cardiovascular vital signs of the user 1214 and the inferred type of activity 1206 to a prescribed medical professional. The measurement reception module 1212 can also use a prediction engine 1216 to analyze the measured cardiovascular vital signs of the user 1214, where the inferred type of activity 1206 is used as a context for this analysis. Then, whenever this analysis detects anomalies in the measured cardiovascular vital signs of the user 1214, the measurement reception module 1212 can output information specifying the detected anomalies 1218.

### 2.0 Other Implementations

While the cardiovascular measurements technique has been described by specific reference to implementations thereof, it is understood that variations and modifications thereof can be made without departing from the true spirit and scope of the cardiovascular measurements technique. For example, rather than the portable computing device being realized in the form of a wearable computing device, an alternate implementation of the cardiovascular measurements technique is possible where the portable computing device is realized in the form of a handheld computing device (such as a specialized smartphone, or a specialized tablet computer, among many other types of handheld computing devices) that is held by the user. Additional types of user activity may be inferred from the received user activity metrics such as the food and/or drink consumption of the user, which can be inferred from the aforementioned environmental images metric, among other ways. The determination of whether or not it is time to measure the cardiovascular vital signs of the user may also be based on one or more previously measured cardiovascular vital signs of the user, or a combination of the inferred type of activity and one or more previously measured cardiovascular vital signs of the user. In addition to measuring one or more of the identified cardiovascular vital signs while the inferred type of activity is occurring, or immediately after the inferred type of activity has completed, or one or more prescribed periods of time after the inferred type of activity has completed, or any combination thereof, one or more cardiovascular vital signs of the user may also be measured on a timed schedule whenever the inferred type of activity is not a “periodic” activity (e.g., whenever the user is stationary and is not exercising).

It is noted that any or all of the aforementioned implementations throughout the description may be used in any combination desired to form additional hybrid implementations. In addition, although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What has been described above includes example implementations. It is, of course, not possible to describe every conceivable combination of components or methodologies for purposes of describing the claimed subject matter, but one of ordinary skill in the art may recognize that many further combinations and permutations are possible. Accordingly, the claimed subject matter is intended to embrace all such alterations, modifications, and variations that fall within the spirit and scope of the appended claims.

In regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the foregoing implementations include a system as well as a computer-readable storage media having computer-executable instructions for performing the acts and/or events of the various methods of the claimed subject matter.

There are multiple ways of realizing the foregoing implementations (such as an appropriate application programming interface (API), tool kit, driver code, operating system, control, standalone or downloadable software object, or the like), which enable applications and services to use the implementations described herein. The claimed subject matter contemplates this use from the standpoint of an API (or other software object), as well as from the standpoint of a software or hardware object that operates according to the implementations set forth herein. Thus, various implementations described herein may have aspects that are wholly in hardware, or partly in hardware and partly in software, or wholly in software.

The aforementioned systems have been described with respect to interaction between several components. It will be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (e.g., hierarchical components).

Additionally, it is noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but generally known by those of skill in the art.

### 3.0 Exemplary Operating Environments

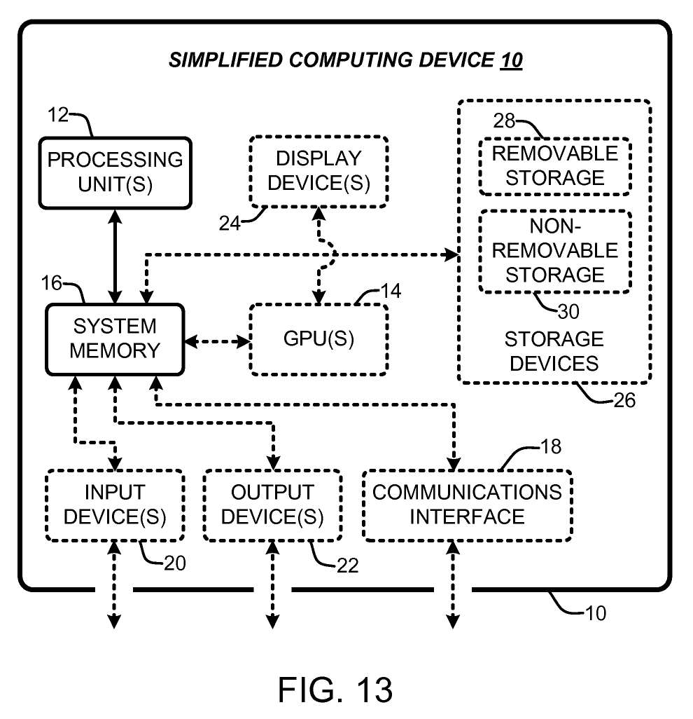

The cardiovascular measurements technique implementations described herein are operational within numerous types of general purpose or special purpose computing system environments or configurations. FIG. 13 illustrates a simplified example of a general-purpose computer system on which various implementations and elements of the cardiovascular measurements technique, as described herein, may be implemented. It is noted that any boxes that are represented by broken or dashed lines in the simplified computing device 10 shown in FIG. 13 represent alternate implementations of the simplified computing device. As described below, any or all of these alternate implementations may be used in combination with other alternate implementations that are described throughout this document. The simplified computing device 10 is typically found in devices having at least some minimum computational capability such as personal computers (PCs), server computers, handheld computing devices, laptop or mobile computers, communications devices such as cell phones and personal digital assistants (PDAs), multiprocessor systems, microprocessor-based systems, set top boxes, programmable consumer electronics, network PCs, minicomputers, mainframe computers, and audio or video media players.

To allow a device to realize the cardiovascular measurements technique implementations described herein, the device should have a sufficient computational capability and system memory to enable basic computational operations. In particular, the computational capability of the simplified computing device 10 shown in FIG. 13 is generally illustrated by one or more processing unit(s) 12, and may also include one or more graphics processing units (GPUs) 14, either or both in communication with system memory 16. Note that that the processing unit(s) 12 of the simplified computing device 10 may be specialized microprocessors (such as a digital signal processor (DSP), a very long instruction word (VLIW) processor, a field-programmable gate array (FPGA), or other micro-controller) or can be conventional central processing units (CPUs) having one or more processing cores.

In addition, the simplified computing device 10 may also include other components, such as, for example, a communications interface 18. The simplified computing device 10 may also include one or more conventional computer input devices 20 (e.g., touchscreens, touch-sensitive surfaces, pointing devices, keyboards, audio input devices, voice or speech-based input and control devices, video input devices, haptic input devices, devices for receiving wired or wireless data transmissions, and the like) or any combination of such devices. The simplified computing device 10 may also include the aforementioned set of user activity sensors (not shown), and primary and secondary ECG electrodes (not shown).

Similarly, various interactions with the simplified computing device 10 and with any other component or feature of the cardiovascular measurements technique implementations described herein, including input, output, control, feedback, and response to one or more users or other devices or systems associated with the cardiovascular measurements technique implementations, are enabled by a variety of Natural User Interface (NUI) scenarios. The NUI techniques and scenarios enabled by the cardiovascular measurements technique implementations include, but are not limited to, interface technologies that allow one or more users user to interact with the cardiovascular measurements technique implementations in a “natural” manner, free from artificial constraints imposed by input devices such as mice, keyboards, remote controls, and the like.

Such NUI implementations are enabled by the use of various techniques including, but not limited to, using NUI information derived from user speech or vocalizations captured via microphones or other sensors (e.g., speech and/or voice recognition). Such NUI implementations are also enabled by the use of various techniques including, but not limited to, information derived from a user's facial expressions and from the positions, motions, or orientations of a user's hands, fingers, wrists, arms, legs, body, head, eyes, and the like, where such information may be captured using various types of 2D or depth imaging devices such as stereoscopic or time-of-flight camera systems, infrared camera systems, RGB (red, green and blue) camera systems, and the like, or any combination of such devices. Further examples of such NUI implementations include, but are not limited to, NUI information derived from touch and stylus recognition, gesture recognition (both onscreen and adjacent to the screen or display surface), air or contact-based gestures, user touch (on various surfaces, objects or other users), hover-based inputs or actions, and the like. Such NUI implementations may also include, but are not limited, the use of various predictive machine intelligence processes that evaluate current or past user behaviors, inputs, actions, etc., either alone or in combination with other NUI information, to predict information such as user intentions, desires, and/or goals. Regardless of the type or source of the NUI-based information, such information may then be used to initiate, terminate, or otherwise control or interact with one or more inputs, outputs, actions, or functional features of the cardiovascular measurements technique implementations described herein.

However, it should be understood that the aforementioned exemplary NUI scenarios may be further augmented by combining the use of artificial constraints or additional signals with any combination of NUI inputs. Such artificial constraints or additional signals may be imposed or generated by input devices such as mice, keyboards, and remote controls, or by a variety of remote or user worn devices such as accelerometers, electromyography (EMG) sensors for receiving myoelectric signals representative of electrical signals generated by user's muscles, heart-rate monitors, galvanic skin conduction sensors for measuring user perspiration, wearable or remote biosensors for measuring or otherwise sensing user brain activity or electric fields, wearable or remote biosensors for measuring user body temperature changes or differentials, and the like. Any such information derived from these types of artificial constraints or additional signals may be combined with any one or more NUI inputs to initiate, terminate, or otherwise control or interact with one or more inputs, outputs, actions, or functional features of the cardiovascular measurements technique implementations described herein.

The simplified computing device 10 may also include other optional components such as one or more conventional computer output devices 22 (e.g., display device(s) 24, audio output devices, video output devices, devices for transmitting wired or wireless data transmissions, and the like). Note that typical communications interfaces 18, input devices 20, output devices 22, and storage devices 26 for general-purpose computers are well known to those skilled in the art, and will not be described in detail herein.

The simplified computing device 10 shown in FIG. 13 may also include a variety of computer-readable media. Computer-readable media can be any available media that can be accessed by the computer 10 via storage devices 26, and can include both volatile and nonvolatile media that is either removable 28 and/or non-removable 30, for storage of information such as computer-readable or computer-executable instructions, data structures, program modules, or other data. Computer-readable media includes computer storage media and communication media. Computer storage media refers to tangible computer-readable or machine-readable media or storage devices such as digital versatile disks (DVDs), blu-ray discs (BD), compact discs (CDs), floppy disks, tape drives, hard drives, optical drives, solid state memory devices, random access memory (RAM), read-only memory (ROM), electrically erasable programmable read-only memory (EEPROM), CD-ROM or other optical disk storage, smart cards, flash memory (e.g., card, stick, and key drive), magnetic cassettes, magnetic tapes, magnetic disk storage, magnetic strips, or other magnetic storage devices. Further, a propagated signal is not included within the scope of computer-readable storage media.

Retention of information such as computer-readable or computer-executable instructions, data structures, program modules, and the like, can also be accomplished by using any of a variety of the aforementioned communication media (as opposed to computer storage media) to encode one or more modulated data signals or carrier waves, or other transport mechanisms or communications protocols, and can include any wired or wireless information delivery mechanism. Note that the terms “modulated data signal” or “carrier wave” generally refer to a signal that has one or more of its characteristics set or changed in such a manner as to encode information in the signal. For example, communication media can include wired media such as a wired network or direct-wired connection carrying one or more modulated data signals, and wireless media such as acoustic, radio frequency (RF), infrared, laser, and other wireless media for transmitting and/or receiving one or more modulated data signals or carrier waves.

Furthermore, software, programs, and/or computer program products embodying some or all of the various cardiovascular measurements technique implementations described herein, or portions thereof, may be stored, received, transmitted, or read from any desired combination of computer-readable or machine-readable media or storage devices and communication media in the form of computer-executable instructions or other data structures. Additionally, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, or media.

The cardiovascular measurements technique implementations described herein may be further described in the general context of computer-executable instructions, such as program modules, being executed by a computing device. Generally, program modules include routines, programs, objects, components, data structures, and the like, that perform particular tasks or implement particular abstract data types. The cardiovascular measurements technique implementations may also be practiced in distributed computing environments where tasks are performed by one or more remote processing devices, or within a cloud of one or more devices, that are linked through one or more communications networks. In a distributed computing environment, program modules may be located in both local and remote computer storage media including media storage devices. Additionally, the aforementioned instructions may be implemented, in part or in whole, as hardware logic circuits, which may or may not include a processor.

Alternatively, or in addition, the functionality described herein can be performed, at least in part, by one or more hardware logic components. For example, and without limitation, illustrative types of hardware logic components that can be used include FPGAs, application-specific integrated circuits (ASICs), application-specific standard products (ASSPs), system-on-a-chip systems (SOCs), complex programmable logic devices (CPLDs), and so on.

### 4.0 Claim Support and Further Implementations

The following paragraphs summarize various examples of implementations which may be claimed in the present document. However, it should be understood that the implementations summarized below are not intended to limit the subject matter which may be claimed in view of the foregoing descriptions. Further, any or all of the implementations summarized below may be claimed in any desired combination with some or all of the implementations described throughout the foregoing description and any implementations illustrated in one or more of the figures, and any other implementations described below. In addition, it should be noted that the following implementations are intended to be understood in view of the foregoing description and figures described throughout this document.

In one implementation, a computer-implemented process is employed for measuring the cardiovascular vital signs of a user. This process uses a computing device to perform the following process actions. More particularly, the process involves receiving one or more user activity metrics from one or more user activity sensors; inferring a type of activity the user is currently engaged in from the received user activity metrics; determining if it is time to measure the cardiovascular vital signs of the user, the determination being based on the inferred type of activity; and whenever it is determined to be time to measure the cardiovascular vital signs of the user, making the measurement.

In one implementation, the process action of making the measurement involves identifying one or more of the cardiovascular vital signs of the user that are to be measured, the identification being based on the inferred type of activity; and measuring the identified cardiovascular vital signs. In one version of this implementation the identified cardiovascular vital signs include a pulse transit time. In another version the identified cardiovascular vital signs include a pulse wave velocity. In another version the identified cardiovascular vital signs include a pulse waveform whose morphology is analyzed to derive various cardiovascular metrics. In another version the identified cardiovascular vital signs include at least one of a heart rate, or heart rate variability. In another version the identified cardiovascular vital signs include at least one of blood pressure, or blood pressure variability. In another version the identified cardiovascular vital signs include at least one of a pulse transit time; or a pulse wave velocity; or a pulse waveform whose morphology is analyzed to derive various cardiovascular metrics; or one or more of a heart rate, or heart rate variability; or one or more of blood pressure, or blood pressure variability.

In one implementation, the process action of measuring the identified cardiovascular vital signs involves measuring one or more of the identified cardiovascular vital signs while the inferred type of activity is occurring. In another implementation this process action involves measuring one or more of the identified cardiovascular vital signs immediately after the inferred type of activity has completed. In another implementation this process action involves measuring one or more of the identified cardiovascular vital signs one or more prescribed periods of time after the inferred type of activity has completed. In another implementation this process action involves at least one of measuring one or more of the identified cardiovascular vital signs while the inferred type of activity is occurring; or measuring one or more of the identified cardiovascular vital signs immediately after the inferred type of activity has completed; or measuring one or more of the identified cardiovascular vital signs one or more prescribed periods of time after the inferred type of activity has completed. In another implementation this process action involves prompting the user to perform an act that initiates the measurement.

The implementations and versions described in any of the previous paragraphs in this section may also be combined with each other, and with one or more of the implementations and versions described prior to this section. For example, some or all of the preceding implementations and versions may be combined with the foregoing implementation where the user activity sensors include one or more of a global positioning system receiver that is attached to the user and is configured to detect the current geographic location of the user, the current altitude of the user, and the current linear velocity of the user; or an accelerometer that is attached to the user and is configured to detect the current acceleration of the user and the direction thereof; or a gyroscope that is attached to the user and is configured to detect the current physical orientation of the user; or a microphone that is either attached to the user or in the immediate vicinity of the user, and is configured to capture sound from the current environment of the user; or a camera that is either attached to the user or in the immediate vicinity of the user, and is configured to capture images from the environment, and to detect the current light-level therein; or an altimeter that is attached to the user and is configured to detect the current altitude of the user; or a wireless communication signal sensor that is attached to the user and is configured to detect the current strength of a wireless communication signal arriving at the user; or a moisture sensor that is attached to the user and is configured to detect moisture in the environment; or a pulse pressure wave sensor that is attached to an arterial site on the body of the user and is configured to measure a pulse pressure wave as it passes by the site.

In addition, some or all of the preceding implementations and versions may be combined with the foregoing implementation where the inferred type of activity includes either walking in a non-stationary manner; or climbing a flight of stairs; or riding in a moving vehicle; or riding in an airplane; or sitting on a stationary object; or sleeping; or lying down but not sleeping; or jogging in a non-stationary manner; or jogging on a treadmill; or walking on the treadmill; or biking; or playing tennis; or golfing; or swimming; or one or more of the food consumption of the user, or the drink consumption of the user. In addition, some or all of the preceding implementations and versions may be combined with the foregoing implementation where the process action of inferring a type of activity the user is currently engaged in from the received user activity metrics involves comparing the received user activity metrics to a set of pre-configured user activity profiles, each of the profiles in the set corresponding to a different type of user activity and specifying either a value or a range of values for each of the user activity metrics that are expected to be received whenever the user is engaged in the different type of user activity; identifying a one of the user activity profiles in the set whose user activity metrics most closely match the received user activity metrics; and assigning the type of user activity that corresponds to the identified user activity profile to be the inferred type of activity.

In addition, some or all of the preceding implementations and versions may be combined with the foregoing implementation where the process action of inferring a type of activity the user is currently engaged in from the received user activity metrics involves computing a confidence metric specifying a level of confidence in the inference; and whenever the confidence metric is less than a prescribed confidence level threshold, prompting the user to specify the type of activity that generated the received user activity metrics. In addition, some or all of the preceding implementations and versions may be combined with the foregoing implementation where the process for measuring the cardiovascular vital signs of a user further involves one or more of the actions of providing one or more of the measured cardiovascular vital signs of the user to the user; or storing the measured cardiovascular vital signs of the user and the inferred type of activity, the inferred type of activity serving as a context for the measured cardiovascular vital signs; or transmitting the measured cardiovascular vital signs of the user and the inferred type of activity to a prescribed medical professional, the inferred type of activity serving as a context for the measured cardiovascular vital signs; or analyzing the measured cardiovascular vital signs of the user using a prediction engine, the inferred type of activity being used as a context for the analysis, and whenever the analysis detects anomalies in the measured cardiovascular vital signs, presenting the user with information specifying the detected anomalies.

In another implementation, a computer-implemented process is employed for measuring the cardiovascular vital signs of a user. This process uses a computing device to perform the following process actions. More particularly, the process involves receiving one or more user activity metrics from one or more user activity sensors; inferring a type of activity the user is currently engaged in from the received user activity metrics; identifying additional context that is associated with the inferred type of activity; determining if it is time to measure the cardiovascular vital signs of the user, this determination being based on both the inferred type of activity and the identified additional context; and whenever it is determined to be time to measure the cardiovascular vital signs of the user, making the measurement. In one version of this implementation, the process action of making the measurement involves identifying one or more of the cardiovascular vital signs of the user that are to be measured, the identification being based on both the inferred type of activity and the identified additional context; and measuring the identified cardiovascular vital signs.

In one implementation, a system for measuring the cardiovascular vital signs of a user includes one or more computing devices and a computer program having program modules executable by the computing devices, where the computing devices are in communication with each other via a computer network whenever there is a plurality of computing devices. The computing devices are directed by the program modules of the computer program to receive one or more user activity metrics; infer a type of activity the user is currently engaged in from the received user activity metrics; determine if it is time to measure the cardiovascular vital signs of the user, this determination being based on the inferred type of activity; whenever it is determined to be time to measure the cardiovascular vital signs of the user, identify one or more of the cardiovascular vital signs of the user that are to be measured and when they are to be measured, this identification being based on the inferred type of activity; output information specifying the cardiovascular vital signs of the user that are to be measured and when they are to be measured; and receive the measured cardiovascular vital signs of the user.

In one implementation, the computing devices are further directed by the program modules of the computer program to perform one or more of the actions of storing the measured cardiovascular vital signs of the user and the inferred type of activity, the inferred type of activity serving as a context for the measured identified cardiovascular vital signs; or transmitting the measured cardiovascular vital signs of the user and the inferred type of activity to a prescribed medical professional, the inferred type of activity serving as a context for the measured cardiovascular vital signs; or analyzing the measured cardiovascular vital signs of the user using a prediction engine, the inferred type of activity being used as a context for the analysis, and whenever the analysis detects anomalies in the measured cardiovascular vital signs of the user, outputting information specifying the detected anomalies.

As indicated previously, the implementations and versions described in any of the previous paragraphs in this section may also be combined with each other, and with one or more of the implementations and versions described prior to this section. For example, some or all of the preceding implementations and versions may be combined with the foregoing implementation where the received user activity metrics include one or more of a user geolocation metric; or a user altitude metric; or a user velocity metric; or a user acceleration metric; or a user physical orientation metric; or an environmental sound metric; or an environmental images metric; or a light-level metric; or a wireless communication signal metric; or a moisture metric; or a pulse waveform from which a user heart rate metric is computed.

In various implementations, a cardiovascular vital signs measurements process is implemented by a step for measuring the cardiovascular vital signs of a user. For example, in one implementation, the cardiovascular vital signs measurements process includes a step for receiving one or more user activity metrics from one or more user activity sensors; a step for inferring a type of activity the user is currently engaged in from the received user activity metrics; a step for determining if it is time to measure the cardiovascular vital signs of the user, the determination being based on the inferred type of activity; and whenever it is determined to be time to measure the cardiovascular vital signs of the user, a step for making the measurement.

In one implementation, the step for making the measurement includes a step for identifying one or more of the cardiovascular vital signs of the user that are to be measured, the identification being based on the inferred type of activity; and a step for measuring the identified cardiovascular vital signs. In one version, the step for measuring the identified cardiovascular vital signs includes at least one of a step for measuring one or more of the identified cardiovascular vital signs while the inferred type of activity is occurring; or a step for measuring one or more of the identified cardiovascular vital signs immediately after the inferred type of activity has completed; or a step for measuring one or more of the identified cardiovascular vital signs one or more prescribed periods of time after the inferred type of activity has completed. In another version, the step for measuring the identified cardiovascular vital signs includes a step for prompting the user to perform an act that initiates the measurement.

In one implementation, the step for inferring a type of activity the user is currently engaged in from the received user activity metrics includes a step for comparing the received user activity metrics to a set of pre-configured user activity profiles, each of the profiles in the set corresponding to a different type of user activity and specifying either a value or a range of values for each of the user activity metrics that are expected to be received whenever the user is engaged in the different type of user activity; a step for identifying a one of the user activity profiles in the set whose user activity metrics most closely match the received user activity metrics; and a step for assigning the type of user activity that corresponds to the identified user activity profile to be the inferred type of activity. In another implementation, the step for inferring a type of activity the user is currently engaged in from the received user activity metrics includes a step for computing a confidence metric specifying a level of confidence in the inference; and whenever the confidence metric is less than a prescribed confidence level threshold, a step for prompting the user to specify the type of activity that generated the received user activity metrics.

In one implementation, the step for measuring the cardiovascular vital signs of a user further includes one or more of a step for providing one or more of the measured cardiovascular vital signs of the user to the user; or a step for storing the measured cardiovascular vital signs of the user and the inferred type of activity, the inferred type of activity serving as a context for the measured cardiovascular vital signs; or a step for transmitting the measured cardiovascular vital signs of the user and the inferred type of activity to a prescribed medical professional, the inferred type of activity serving as a context for the measured cardiovascular vital signs; or a step for analyzing the measured cardiovascular vital signs of the user using a prediction engine, the inferred type of activity being used as a context for the analysis, and whenever the analysis detects anomalies in the measured cardiovascular vital signs, a step for presenting the user with information specifying the detected anomalies.

In another implementation, the cardiovascular vital signs measurements process includes a step for receiving one or more user activity metrics from one or more user activity sensors; a step for inferring a type of activity the user is currently engaged in from the received user activity metrics; a step for identifying additional context that is associated with the inferred type of activity; a step for determining if it is time to measure the cardiovascular vital signs of the user, the determination being based on both the inferred type of activity and the identified additional context; and whenever it is determined to be time to measure the cardiovascular vital signs of the user, a step for making the measurement. In one version of this implementation, the step for making the measurement includes a step for identifying one or more of the cardiovascular vital signs of the user that are to be measured, this identification being based on both the inferred type of activity and the identified additional context; and a step for measuring the identified cardiovascular vital signs.

In one implementation, a cardiovascular vital signs measurements system is implemented by means for measuring the cardiovascular vital signs of a user. For example, in one implementation, the cardiovascular vital signs measurements system includes one or more computing devices, the computing devices being in communication with each other via a computer network whenever there is a plurality of computing devices, the computing devices including processors configured to execute a metrics receiving step for receiving one or more user activity metrics; an inference step for inferring a type of activity the user is currently engaged in from the received user activity metrics; a determination step for determining if it is time to measure the cardiovascular vital signs of the user, the determination being based on the inferred type of activity; whenever it is determined to be time to measure the cardiovascular vital signs of the user, an identification step for identifying one or more of the cardiovascular vital signs of the user that are to be measured and when they are to be measured, the identification being based on the inferred type of activity; a measurement information outputting step for outputting information specifying the cardiovascular vital signs of the user that are to be measured and when they are to be measured; and a vital signs receiving step for receiving the measured cardiovascular vital signs of the user.

In one implementation, the processors are further configured to execute one or more of a storing step for storing the measured cardiovascular vital signs of the user and the inferred type of activity, the inferred type of activity serving as a context for the measured identified cardiovascular vital signs; or a transmitting step for transmitting the measured cardiovascular vital signs of the user and the inferred type of activity to a prescribed medical professional, the inferred type of activity serving as a context for the measured cardiovascular vital signs; or an analysis step for analyzing the measured cardiovascular vital signs of the user using a prediction engine, the inferred type of activity being used as a context for the analysis, and whenever the analysis detects anomalies in the measured cardiovascular vital signs of the user, an anomalies outputting step for outputting information specifying the detected anomalies.

Wherefore, what is claimed is:

1. A computer-implemented process for measuring the cardiovascular vital signs of a user, comprising the actions of:
using a computing device to perform the following process actions:
receiving one or more user activity metrics from one or more user activity sensors;
inferring a type of activity the user is currently engaged in from the received user activity metrics;
determining if it is time to measure the cardiovascular vital signs of the user, said determination being based on the inferred type of activity; and
whenever it is determined to be time to measure the cardiovascular vital signs of the user, making said measurement.

2. The process of claim 1, wherein the process action of making said measurement comprises the actions of:
identifying one or more of the cardiovascular vital signs of the user that are to be measured, said identification being based on the inferred type of activity; and
measuring the identified cardiovascular vital signs.

3. The process of claim 2, wherein the identified cardiovascular vital signs comprise a pulse transit time.

4. The process of claim 2, wherein the identified cardiovascular vital signs comprise a pulse wave velocity.

5. The process of claim 2, wherein the identified cardiovascular vital signs comprise a pulse waveform whose morphology is analyzed to derive various cardiovascular metrics.

6. The process of claim 2, wherein the identified cardiovascular vital signs comprise at least one of:
a heart rate; or
heart rate variability.

7. The process of claim 2, wherein the identified cardiovascular vital signs comprise at least one of:
blood pressure; or
blood pressure variability.

8. The process of claim 2, wherein the process action of measuring the identified cardiovascular vital signs comprises an action of measuring one or more of the identified cardiovascular vital signs while the inferred type of activity is occurring.

9. The process of claim 2, wherein the process action of measuring the identified cardiovascular vital signs comprises an action of measuring one or more of the identified cardiovascular vital signs immediately after the inferred type of activity has completed.

10. The process of claim 2, wherein the process action of measuring the identified cardiovascular vital signs comprises an action of measuring one or more of the identified cardiovascular vital signs one or more prescribed periods of time after the inferred type of activity has completed.

11. The process of claim 2, wherein the process action of measuring the identified cardiovascular vital signs comprises an action of prompting the user to perform an act that initiates said measurement.

12. The process of claim 1, wherein the user activity sensors comprise one or more of:
a global positioning system receiver that is attached to the user and is configured to detect the current geographic location of the user, the current altitude of the user, and the current linear velocity of the user; or
an accelerometer that is attached to the user and is configured to detect the current acceleration of the user and the direction thereof; or
a gyroscope that is attached to the user and is configured to detect the current physical orientation of the user; or
a microphone that is either attached to the user or in the immediate vicinity of the user, and is configured to capture sound from the current environment of the user; or
a camera that is either attached to the user or in the immediate vicinity of the user, and is configured to capture images from said environment, and to detect the current light-level therein; or
an altimeter that is attached to the user and is configured to detect the current altitude of the user; or
a wireless communication signal sensor that is attached to the user and is configured to detect the current strength of a wireless communication signal arriving at the user; or
a moisture sensor that is attached to the user and is configured to detect moisture in said environment; or
a pulse pressure wave sensor that is attached to an arterial site on the body of the user and is configured to measure a pulse pressure wave as it passes by said site.

13. The process of claim 1, wherein the inferred type of activity comprises either:
walking in a non-stationary manner; or
climbing a flight of stairs; or
riding in a moving vehicle; or
riding in an airplane; or
sitting on a stationary object; or
sleeping; or
lying down but not sleeping; or
jogging in a non-stationary manner; or
jogging on a treadmill; or
walking on the treadmill; or
biking; or
playing tennis; or
golfing; or
swimming; or
one or more of the food consumption of the user, or the drink consumption of the user.

14. The process of claim 1, wherein the process action of inferring a type of activity the user is currently engaged in from the received user activity metrics comprises the actions of:
comparing the received user activity metrics to a set of pre-configured user activity profiles, each of the profiles in said set corresponding to a different type of user activity and specifying either a value or a range of values for each of the user activity metrics that are expected to be received whenever the user is engaged in said different type of user activity;
identifying a one of the user activity profiles in said set whose user activity metrics most closely match the received user activity metrics; and
assigning the type of user activity that corresponds to the identified user activity profile to be the inferred type of activity.

15. The process of claim 1, wherein the process action of inferring a type of activity the user is currently engaged in from the received user activity metrics comprises the actions of:
computing a confidence metric specifying a level of confidence in said inference; and
whenever the confidence metric is less than a prescribed confidence level threshold, prompting the user to specify the type of activity that generated the received user activity metrics.

16. The process of claim 1, further comprising one or more of the actions of:
providing one or more of the measured cardiovascular vital signs of the user to the user; or
storing the measured cardiovascular vital signs of the user and the inferred type of activity, the inferred type of activity serving as a context for said measured cardiovascular vital signs; or
transmitting the measured cardiovascular vital signs of the user and the inferred type of activity to a prescribed medical professional, the inferred type of activity serving as a context for said measured cardiovascular vital signs; or
analyzing the measured cardiovascular vital signs of the user using a prediction engine, the inferred type of activity being used as a context for said analysis, and
whenever said analysis detects anomalies in said measured cardiovascular vital signs, presenting the user with information specifying the detected anomalies.

17. A computer-implemented process for measuring the cardiovascular vital signs of a user, comprising the actions of:
using a computing device to perform the following process actions:
receiving one or more user activity metrics from one or more user activity sensors;
inferring a type of activity the user is currently engaged in from the received user activity metrics;
identifying additional context that is associated with the inferred type of activity;
determining if it is time to measure the cardiovascular vital signs of the user, said determination being based on both the inferred type of activity and the identified additional context; and
whenever it is determined to be time to measure the cardiovascular vital signs of the user, making said measurement.

18. The process of claim 17, wherein the process action of making said measurement comprises the actions of:
identifying one or more of the cardiovascular vital signs of the user that are to be measured, said identification being based on both the inferred type of activity and the identified additional context; and
measuring the identified cardiovascular vital signs.

19. A system for measuring the cardiovascular vital signs of a user, comprising:
one or more computing devices, said computing devices being in communication with each other via a computer network whenever there is a plurality of computing devices; and
a computer program having program modules executable by said computing devices, said computing devices being directed by the program modules of the computer program to,
receive one or more user activity metrics;
infer a type of activity the user is currently engaged in from the received user activity metrics;
determine if it is time to measure the cardiovascular vital signs of the user, said determination being based on the inferred type of activity;
whenever it is determined to be time to measure the cardiovascular vital signs of the user, identify one or more of the cardiovascular vital signs of the user that are to be measured and when they are to be measured, said identification being based on the inferred type of activity;
output information specifying the cardiovascular vital signs of the user that are to be measured and when they are to be measured; and
receive the measured cardiovascular vital signs of the user.

20. The system of claim 19, wherein said computing devices are further directed by the program modules of the computer program to perform one or more of the actions of:
storing the measured cardiovascular vital signs of the user and the inferred type of activity, the inferred type of activity serving as a context for the measured identified cardiovascular vital signs; or
transmitting the measured cardiovascular vital signs of the user and the inferred type of activity to a prescribed medical professional, the inferred type of activity serving as a context for said measured cardiovascular vital signs; or
analyzing the measured cardiovascular vital signs of the user using a prediction engine, the inferred type of activity being used as a context for said analysis, and
whenever said analysis detects anomalies in the measured cardiovascular vital signs of the user, outputting information specifying the detected anomalies.
